(19) United States
(12) Patent Application Publication
(10) Pub. No.: US 2023/0047587 A1
(43) Pub. Date: Feb. 16, 2023

(54) APPARATUS AND METHOD FOR ESTIMATING BEHAVIOR OF USER BASED ON IMAGE CONVERTED FROM SENSING DATA, AND METHOD FOR CONVERTING SENSING DATA INTO IMAGE

(71) Applicant: ELECTRONICS AND TELECOMMUNICATIONS RESEARCH INSTITUTE, Daejeon (KR)

(72) Inventors: Min-Gi JEONG, Daejeon (KR); Kang-Bok LEE, Daejeon (KR); Sang-Yeoun LEE, Daejeon (KR); Ji-Hun JEON, Daejeon (KR)

(73) Assignee: ELECTRONICS AND TELECOMMUNICATIONS RESEARCH INSTITUTE, Daejeon (KR)

(21) Appl. No.: 17/516,130

(22) Filed: Nov. 1, 2021

(30) Foreign Application Priority Data
Aug. 10, 2021 (KR) 10-2021-0104946

(51) Int. Cl. A61B 5/11; G06K 9/00

(52) U.S. Cl. CPC A61B 5/1128; G06K 9/00348; G06K 9/00771; A61B 5/1116; A61B 5/1121

(57) ABSTRACT

Disclosed herein are an apparatus and a method for estimating the behavior of a user based on an image converted from sensing data. The apparatus for estimating the behavior of a user based on an image converted from sensing data includes memory for storing at least one program, and a processor for executing the program, wherein the program performs acquiring sensing data measured by one or more behavior measurement devices worn by the user, converting sensing data of the user obtained for a predetermined time period into images, and estimating the behavior of the user from the images of the user based on a pre-trained model.

### CROSS REFERENCE TO RELATED APPLICATION

This application claims the benefit of Korean Patent Application No. 10-2021-0104946, filed Aug. 10, 2021, which is hereby incorporated by reference in its entirety into this application.

### BACKGROUND OF THE INVENTION

### 1. Field

The following embodiments relate to technology for analyzing the behavior of a user.

### 2. Description of Related Art

Existing technology for analyzing the behavior (motion) of a pedestrian includes a method using markers or imaging cameras and a method for attaching inertial devices to a human body.

It is difficult to utilize the method using markers or imaging cameras in daily life due to spatial limitations and difficulty in installation. Further, the method for attaching inertial devices to a human body may be configured to extract features from data, obtained by measuring acceleration values depending on a time axis, and to determine behavior, and may limitedly analyze the behavior of a user depending only on defined behavior types and schemes for reproducing the motion of the body structure of the user.

Therefore, the existing methods have a difficulty in that feature values and criteria are differently applied depending on various environments and situations, and cannot accurately identify various patterns that may appear in the same type of behavior.

### SUMMARY OF THE INVENTION

An embodiment is intended to accurately identify the behavior of a user even in various environments and situations.

An embodiment is intended to accurately identify the behavior of a user depending on various patterns appearing in the same type of behavior.

In accordance with an aspect, there is provided an apparatus for estimating a behavior of a user based on an image converted from sensing data, including memory for storing at least one program, and a processor for executing the program, wherein the program performs acquiring sensing data measured by one or more behavior measurement devices worn by the user, converting sensing data of the user obtained for a predetermined time period into images, and estimating the behavior of the user from the images of the user based on a pre-trained model.

The sensing data of the user obtained for the predetermined time period may be measured during a predetermined time before and after a time point at which an event, an intensity of an impact of which is equal to or greater than a predetermined threshold value, occurred.

The program may further perform, upon converting the sensing data into the images, generating a primary image for each of one or more colors based on the sensing data, and when there are multiple primary images, generating one secondary image by combining primary images generated for each of two or more colors.

The program may further perform, upon generating the primary image for each of the one or more colors, when there are multiple behavior measurement devices, generating image tables in which pixel values, calculated based on pieces of sensing data measured through respective multiple behavior measurement devices, are recorded, and converting the generated image tables into primary images in different colors.

The program may further perform, upon generating the primary image for each of the one or more colors, when there are multiple behavior measurement devices, generating multiple image tables in which pixel values, calculated by combining pieces of sensing data measured through the behavior measurement devices with each other, are recorded, and converting the generated image tables into primary images in different colors.

The sensing data may include acceleration values on an x axis, a y axis, and a z axis over time for each of one or more behavior measurement devices worn on different body regions of the user, and the program may be configured to, upon generating the primary image for each of the one or more colors, when each image is a two-dimensional (2D) image, convert a 2D image table into a primary image, wherein each pixel value of the 2D image table is determined to be any one of a geometric average, a maximum value, and a minimum value of one or more of acceleration values on the x axis, the y axis, and the z axis over time, measured through the one or more behavior measurement devices.

The sensing data may include acceleration values on an x axis, a y axis, and a z axis over time for each of one or more behavior measurement devices worn on different body regions of the user, and the program may be configured to, upon generating the primary image for each of the one or more colors, when each image is a three-dimensional (3D) image, convert a 3D image table into a primary image, wherein each pixel value of the 3D image table is determined to be a value calculated based on the acceleration values on the x axis, the y axis and the z axis over time, measured through the one or more behavior measurement devices.

The program may further perform, upon estimating the behavior of the user, determining based on the images whether the behavior of the user is in a normal or abnormal state, and if it is determined that the behavior the user is in an abnormal state, reporting a dangerous situation.

In accordance with another aspect, there is provided a method for estimating a behavior of a user based on an image converted from sensing data, including acquiring sensing data measured by one or more behavior measurement devices worn by the user, converting sensing data of the user obtained for a predetermined time period into images, and estimating the behavior of the user from the images of the user based on a pre-trained model.

The sensing data of the user obtained for the predetermined time period may be measured during a predetermined time before and after a time point at which an event, an intensity of an impact of which is equal to or greater than a predetermined threshold value, occurred.

Converting the sensing data into the images may include generating a primary image for each of one or more colors based on the sensing data, and when there are multiple primary images, generating one secondary image by combining primary images generated for each of two or more colors.

Generating the primary image for each of the one or more colors may include, when there are multiple behavior measurement devices, generating image tables in which pixel values, calculated based on pieces of sensing data measured through respective multiple behavior measurement devices, are recorded, and converting the generated image tables into primary images in different colors.

Generating the primary image for each of the one or more colors may include, when there are multiple behavior measurement devices, generating multiple image tables in which pixel values, calculated by combining pieces of sensing data measured through the behavior measurement devices with each other, are recorded, and converting the generated image tables into primary images in different colors.

The sensing data may include acceleration values on an x axis, a y axis, and a z axis over time for each of one or more behavior measurement devices worn on different body regions of the user, and generating the primary image for each of the one or more colors may be configured to, when each image is a two-dimensional (2D) image, convert a 2D image table into a primary image, wherein each pixel value of the 2D image table is determined to be any one of a geometric average, a maximum value, and a minimum value of one or more of acceleration values on the x axis, the y axis, and the z axis over time, measured through the one or more behavior measurement devices.

The sensing data may include acceleration values on an x axis, a y axis, and a z axis over time for each of one or more behavior measurement devices worn on different body regions of the user, and generating the primary image for each of the one or more colors may be configured to, when each image is a three-dimensional (3D) image, convert a 3D image table into a primary image, wherein each pixel value of the 3D image table is determined to be a value calculated based on the acceleration values on the x axis, the y axis and the z axis over time, measured through the one or more behavior measurement devices.

In accordance with a further aspect, there is provided a method for converting sensing data into an image, including generating a primary image for each of one or more colors based on sensing data of a user obtained for a predetermined time period, and when there are multiple primary images, generating one secondary image by combining primary images generated for each of two or more colors.

Generating the primary image for each of one or more colors may include, when the sensing data is acquired from multiple behavior measurement devices, generating image tables in which pixel values, calculated based on pieces of sensing data measured through respective multiple behavior measurement devices, are recorded, and converting the generated image tables into primary images in different colors.

Generating the primary image for each of one or more colors may include, when the sensing data is acquired from multiple behavior measurement devices, generating multiple image tables in which pixel values, calculated by combining pieces of sensing data measured through the behavior measurement devices with each other, are recorded, and converting the generated image tables into primary images in different colors.

The sensing data may include acceleration values on an x axis, a y axis, and a z axis over time for each of one or more behavior measurement devices worn on different body regions of the user, and generating the primary image for each of the one or more colors may be configured to, when each image is a two-dimensional (2D) image, convert a 2D image table into a primary image, wherein each pixel value of the 2D image table is determined to be any one of a geometric average, a maximum value, and a minimum value of one or more of acceleration values on the x axis, the y axis, and the z axis over time, measured through the one or more behavior measurement devices.

The sensing data may include acceleration values on an x axis, a y axis, and a z axis over time for each of one or more behavior measurement devices worn on different body regions of the user, and generating the primary image for each of the one or more colors may be configured to, when each image is a three-dimensional (3D) image, convert a 3D image table into a primary image, wherein each pixel value of the 3D image table is determined to be a value calculated based on the acceleration values on the x axis, the y axis and the z axis over time, measured through the one or more behavior measurement devices.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a schematic block configuration diagram of a system for estimating the behavior of a user based on an image converted from sensing data according to an embodiment;

FIG. 2 is a flowchart illustrating the operation of a behavior measurement device according to an embodiment;

FIG. 3 is a flowchart illustrating the operation of a user behavior estimation apparatus according to an embodiment;

FIG. 4 is a flowchart illustrating in detail the step of converting sensing data into an image according to an embodiment;

FIG. 5 is a diagram illustrating an example of 2D image tables according to an embodiment, and FIG. 6 is a diagram illustrating an example of 2D image generation according to an embodiment;

FIGS. 7 and 8 are diagrams illustrating examples of image tables when motion types are different from each other according to embodiments;

FIG. 9 is a diagram illustrating an example of 2D image tables according to another embodiment;

FIG. 10 is a diagram illustrating an example of 3D image tables according to a further embodiment; and

FIG. 11 is a diagram illustrating the configuration of a computer system according to an embodiment.

### DESCRIPTION OF THE PREFERRED EMBODIMENTS

Advantages and features of the present invention and methods for achieving the same will be clarified with reference to embodiments described later in detail together with the accompanying drawings. However, the present invention is capable of being implemented in various forms, and is not limited to the embodiments described later, and these embodiments are provided so that this invention will be thorough and complete and will fully convey the scope of the present invention to those skilled in the art. The present invention should be defined by the scope of the accompanying claims. The same reference numerals are used to designate the same components throughout the specification.

It will be understood that, although the terms “first” and “second” may be used herein to describe various components, these components are not limited by these terms. These terms are only used to distinguish one component from another component. Therefore, it will be apparent that a first component, which will be described below, may alternatively be a second component without departing from the technical spirit of the present invention.

The terms used in the present specification are merely used to describe embodiments, and are not intended to limit the present invention. In the present specification, a singular expression includes the plural sense unless a description to the contrary is specifically made in context. It should be understood that the term “comprises” or “comprising” used in the specification implies that a described component or step is not intended to exclude the possibility that one or more other components or steps will be present or added.

Unless differently defined, all terms used in the present specification can be construed as having the same meanings as terms generally understood by those skilled in the art to which the present invention pertains. Further, terms defined in generally used dictionaries are not to be interpreted as having ideal or excessively formal meanings unless they are definitely defined in the present specification.

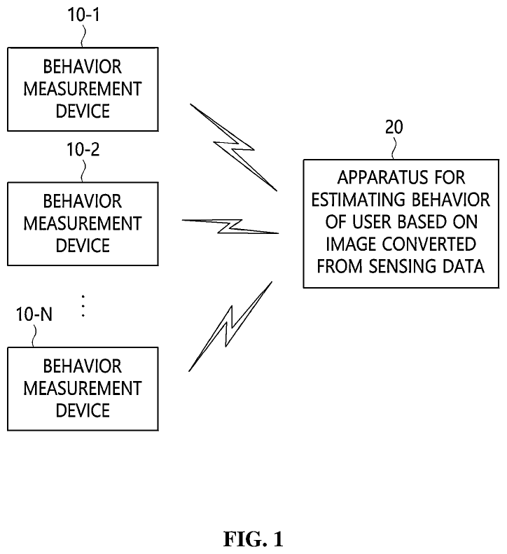

Hereinafter, an apparatus and method for estimating the behavior of a user based on an image converted from sensing data and a device for converting sensing data into an image according to embodiments will be described in detail with reference to FIGS. 1 to 11.

FIG. 1 is a schematic block configuration diagram of a system for estimating the behavior of a user based on an image converted from sensing data according to an embodiment.

Referring to FIG. 1, a system 1 for estimating the behavior of a user based on an image converted from sensing data according to an embodiment may be implemented in a form in which one or more behavior measurement devices 10-1, 10-2, . . . , 10-N and an apparatus 20 for estimating the behavior of a user based on an image converted from sensing data (hereinafter referred to as a “user behavior estimation apparatus 20”) are operated in conjunction with each other through wired communication.

The one or more behavior measurement devices 10-1, 10-2, . . . , 10-N may be attached to part of the user's body to sense the behavior of the user, and may transmit sensed behavior information to the user behavior estimation apparatus 20 in a wireless manner.

Here, the part of the user's body may be at least one of, for example, the waist and feet of the user, and the one or more behavior measurement devices 10-1, 10-2, . . . , 10-N may be implemented in a form easily attachable to the belt on the waist or the soles of shoes.

Here, the one or more behavior measurement devices 10-1, 10-2, . . . , 10-N may include a sensor for sensing the behavior of the user. For example, an inertial sensor or the like may be included in the sensor. Therefore, the sensing data may include respective acceleration values on an x axis, a y axis, and a z axis depending on the motion of the parts of the user's body on which the behavior measurement devices 10-1, 10-2, . . . , 10-N are worn. However, these values are only examples, and the sensing data of the present invention is not limited to such acceleration values. That is, it is noted that other types of sensing data with which the behavior of the user can be analyzed may be applied to the embodiment of the present invention.

Also, each of the one or more behavior measurement devices 10-1, 10-2, . . . , 10-N may include a communication unit which can transmit the sensing data, obtained by measuring the behavior of the user using the sensor, to the user behavior estimation apparatus 20.

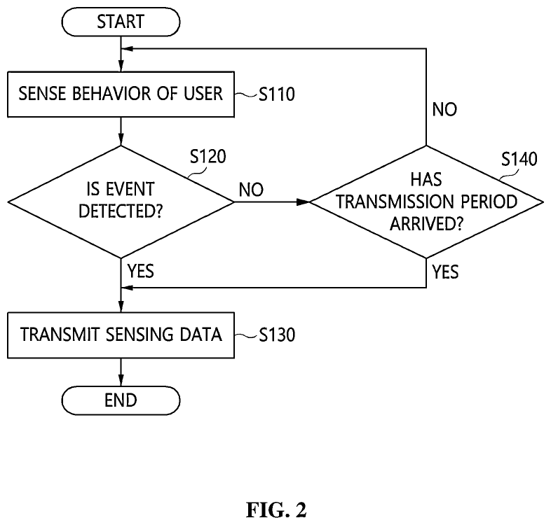

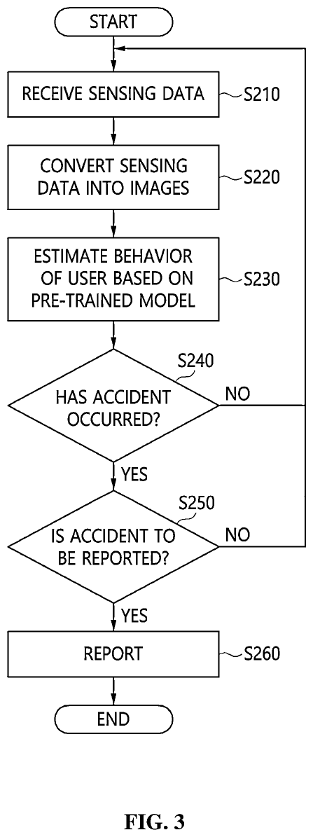

Further, each of the one or more behavior measurement devices 10-1, 10-2, . . . , 10-N may include memory, which stores the sensing data, and a control unit which controls an operation of transmitting the sensing data, stored in the memory, to the user behavior estimation apparatus 20 through the communication unit either upon occurrence of an event or at intervals of a predetermined period. The detailed operation of the control unit of each of the behavior measurement devices 10-1, 10-2, . . . , 10-N according to the embodiment will be described later with reference to FIG. 2.

Meanwhile, the user behavior estimation apparatus 20 may convert the sensing data transmitted from the one or more behavior measurement devices 10-1, 10-2, . . . , 10-N into images, may then analyze the behavior of the user from the images, and may respond to the analyzed behavior.

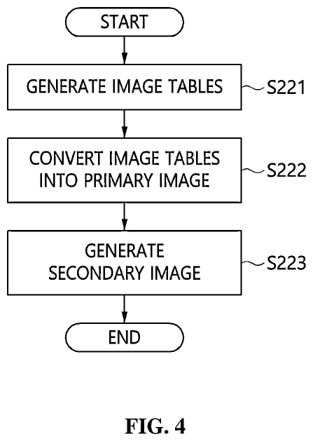

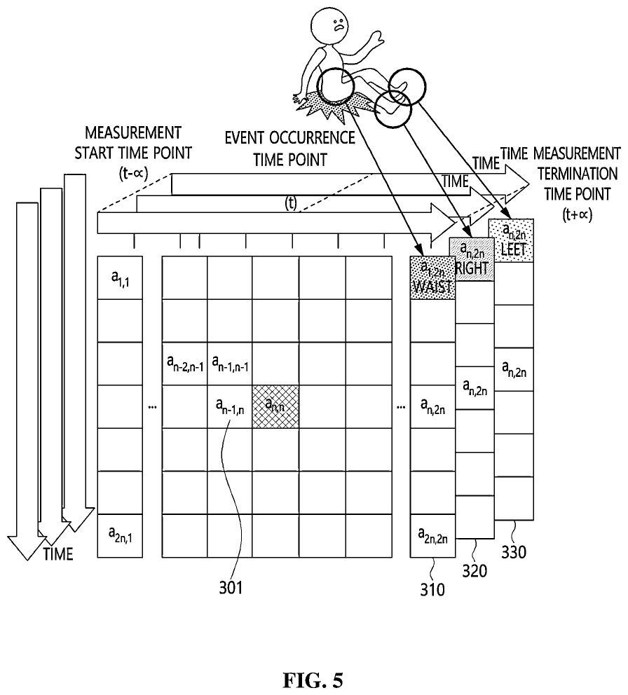

Such a user behavior estimation apparatus 20 may be a mobile terminal itself possessed by the user, or may be an application installed on the mobile terminal of the user. The detailed operation of the user behavior estimation apparatus 20 according to the embodiment will be described later with reference to FIGS. 3 and 4.

FIG. 2 is a flowchart illustrating the operation of a behavior measurement device according to an embodiment.

Referring to FIG. 2, each of one or more behavior measurement devices 10-1, 10-2, . . . , 10-N may sense a behavior in the region of a user on which the corresponding behavior measurement device is worn at step S110.

Here, sensing data may be stored together with the time point at which measurement is performed. For example, when a corresponding one of the behavior measurement devices 10-1, 10-2, . . . , 10-N is an inertial sensor, the sensing data may include information about the measurement time point and acceleration values on an x axis, a y axis, and a z axis depending on the motion of the corresponding body region of the user at the measurement time point.

While step S110 is being performed, the corresponding one of the behavior measurement devices 10-1, 10-2, . . . , 10-N detects whether an event has occurred at step S120.

Here, whether an event has occurred may be determined depending on whether the intensity of an impact applied to the corresponding one of the behavior measurement devices 10-1, 10-2, . . . , 10-N is equal to or greater than a predetermined threshold value. Here, examples of the event may include jumping in place, bumping against a wall, falling, etc.

If, as a result of the detection at step S120, it is determined that an event has occurred, the corresponding one of the behavior measurement devices 10-1, 10-2, . . . , 10-N transmits the sensing data, obtained for a predetermined time period, to the user behavior estimation apparatus 20 at step S130.

In contrast, if, as a result of the detection at step S120, it is determined that no event has occurred, the corresponding one of the behavior measurement devices 10-1, 10-2, . . . , 10-N checks whether a transmission period has arrived at step S140.

When, as a result of the checking at step S140, it is determined that the transmission period has arrived, the corresponding one of the behavior measurement devices 10-1, 10-2, . . . , 10-N performs step S130. That is, when no event occurs, the corresponding behavior measurement device transmits the sensing data to the user behavior estimation apparatus 20 at intervals of a predetermined period.

In contrast, when, as a result of the checking at step S140, it is determined that a transmission period has not arrived, the corresponding one of the behavior measurement devices 10-1, 10-2, . . . , 10-N continues to perform step S110.

FIG. 3 is a flowchart illustrating the operation of a user behavior estimation apparatus according to an embodiment. Meanwhile, the details of a method for estimating the behavior of a user based on an image converted from sensing data according to the embodiment are identical to those of the operation of the user behavior estimation apparatus, which will be described later, and thus detailed descriptions thereof will be omitted.

Referring to FIG. 3, the user behavior estimation apparatus 20 receives sensing data, measured by one or more behavior measurement devices 10-1, 10-2, . . . , 10-N, worn by a user, at step S210.

Here, the sensing data of the user obtained for a predetermined time period may be data that is measured during a predetermined time before and after the time point at which an event, the intensity of an impact of which is equal to or greater than a predetermined threshold value, occurred, or that is measured during a predetermined transmission period.

Thereafter, the user behavior estimation apparatus 20 converts the sensing data of the user, obtained for the predetermined time period, into images at step S220.

In this case, when the sensing data is converted into the images according to the embodiment, the values of the collected sensing data are reflected in the images without change, thus preventing pieces of important information that influence accidents from being omitted. Further, not only measurement values over time but also information in a frequency domain may be reflected in the images, because relationships between sensing data values in the directions of different axes before and after the time point at which the event occurred, sensing data values in different regions, and measurement values at different times may be converted into images. The details of step S220 will be described later with reference to FIG. 4.

The user behavior estimation apparatus 20 estimates the behavior of the user from the converted images based on a previously trained model at step S230.

Here, at step S230, the behavior of the user may be inferred from images converted from sensing data related to various types of motion based on a previously trained deep-learning model. Here, the deep-learning model may be designed as any of various neural network algorithms including a Convolutional Neural Network (CNN).

As described above, when the behavior of the user is inferred from the converted images based on the deep-learning model, various response services may be performed using the results of the inference. For example, when an accident, such as a falling accident, dropping, or bumping, which may occur during walking, occurs, a service for promptly responding to such an accident may be performed.

Referring to FIG. 3, the user behavior estimation apparatus 20 may determine whether the estimated behavior of the user is a motion corresponding to the accident at step S240. That is, when falling, dropping or bumping by the user occurs, values measured by an acceleration sensor may differ from values measured during normal walking, and thus it may be determined that an abnormal state has occurred.

If it is determined at step S240 that no accident has occurred, the user behavior estimation apparatus 20 repeatedly performs steps S210 to S230.

In contrast, if it is determined at step S240 that an accident has occurred, the user behavior estimation apparatus 20 determines whether to report the occurrence of the accident at step S250.

If it is determined at step S240 that the behavior of the user is motion corresponding to an accident, the user behavior estimation apparatus 20 may determine whether to report the corresponding accident at step S250. For example, if the user falls down on the street, whether the accident is to be reported may be determined depending on the result of determining whether the severity of the accident is sufficient to report the accident, or the like.

If it is determined at step S250 that it is not required to report the accident, the user behavior estimation apparatus 20 returns to step S210.

In contrast, if it is determined at step S250 that it is required to report the accident, the user behavior estimation apparatus 20 automatically reports the occurrence of the accident at step S260. That is, a report of the occurrence of the accident to a pre-stored phone number is made. Here, the pre-stored phone number may be that of a police station, a hospital, a guardian, or the like.

However, steps S240 to S260 indicate only an example of a service that utilizes the results of estimation of the behavior of the user, and the present invention is not limited thereto. That is, it is noted that the results of estimating the behavior of the user at steps S210 to S230 may also be utilized in various other services.

FIG. 4 is a flowchart illustrating in detail step S220 of converting sensing data into images according to an embodiment. Meanwhile, details of an apparatus and a method for converting sensing data into an image according to embodiments are identical to those of step S220 of converting sensing data into images, which will be described later, and thus separate detailed descriptions thereof will be omitted.

Referring to FIG. 4, step S220 of converting sensing data into images may include steps S221 and S222 of generating a primary image for each of one or more colors based on the sensing data, and step S223 of, when there are multiple primary images, generating one secondary image by combining respective primary images generated for two or more colors.

Here, steps S221 and S222 of generating the primary image for each of one or more colors based on the sensing data may include step S221 of generating image tables in which pixel values calculated based on the sensing data are recorded and step S222 of converting each of the generated image tables into primary images in different colors.

Here, at step S221 of generating the image tables in which pixel values calculated based on the sensing data are recorded, each of the image tables may be generated as an image table corresponding to at least one of three colors, namely red, green, and blue.

Meanwhile, step S220 of converting the sensing data into the images may be implemented in various embodiments depending on the number of behavior measurement devices through which the sensing data is acquired.

Further, step S220 of converting the sensing data into the images may be implemented in various embodiments depending on whether each image to be generated is a two-dimensional (2D) image or a three-dimensional (3D) image.

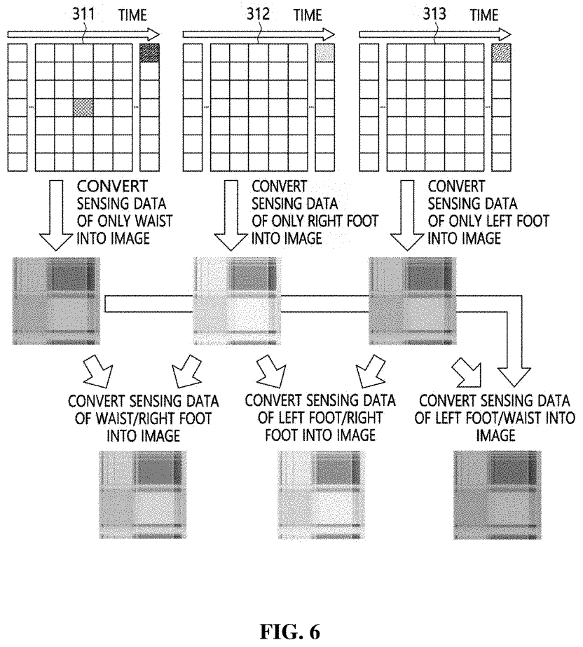

To aid in understanding of the present invention, an example in which a 2D image is generated using sensing data acquired in the state in which the user wears the behavior measurement devices 10-1, 10-2, . . . , 10-N on his or her waist, left foot, and right foot is described below with reference to FIGS. 5 to 8.

FIG. 5 is a diagram illustrating an example of 2D image tables according to an embodiment, and FIG. 6 is a diagram illustrating an example of 2D image generation according to an embodiment.

Referring to FIG. 5, when multiple behavior measurement devices 10-1, 10-2, . . . , 10-N are attached to the waist, left foot, and right foot, respectively, sensing data acquired through the behavior measurement device attached to the waist may be used to generate a red image table 310, sensing data acquired through the behavior measurement device attached to the right foot may be used to generate a green image table 320, and sensing data acquired through the behavior measurement device attached to the left foot may be used to generate a blue image table 330.

Meanwhile, the sensing data that is the target of image conversion may be collected during a certain time period α before and after the time point t at which an event occurred. That is, the sensing data may be regarded as sensing data measured during the time period from the time point t−α to the time point t+α.

At this time, the number 2n of pieces of sensing data measured during the period from the time point t−α to the time point t+α may be calculated using the following Equation (1):

2n=2α*(sampling rate) (1)

In Equation (1), the sampling rate may be the number of pieces of sensing data collected per second.

Also, each of the number of rows and the number of columns in each image table may be the number 2n of pieces of sensing data over time. That is, referring to FIG. 5, the corresponding image table may be composed of 2n×2n pixels from pixel a_{1,1} to pixel a_{2n,2n}.

In the 2n×2n pixels of each of the image tables 310 to 330, pixel values based on the acquired sensing data may be calculated and recorded.

At this time, when the pixel values recorded in the image tables 310 to 330 are calculated, relationships between pieces of sensing data at different times may be calculated, and may then be reflected in the pixel values.

That is, the value a_{n,n} of one pixel in the image table 310 may be defined as a function taking as variables the row x and the column y of the pixel represented by the following Equation (2).

a_{n,n}=F(x,y) (2)

In Equation (2), the values of row x and column y may be defined as respective functions based on acceleration values (ACC_{waist_x axis}, ACC_{waist_y axis}, and ACC_{waist_z axis}) at time t, as represented by the following Equation (3):

u(t)=x

v(t)=y (3)

In Equation (3), each of u(t) and v(t) may be defined in various embodiments. In accordance with an embodiment, u(t) and v(t) may be defined as acceleration values at time t for one or more of x, y, and z axes of an inertial sensor. For example, u(t) may be defined as ACC_{waist_x axis_t}, and v(t) may be defined as ACC_{waist_y axis_t}.

Therefore, the value a_{n,n} of one pixel of the image table 310 may be calculated using the function F, as shown in Equation (2), which exploits ACC_{waist_x axis_t} as the variable of the row corresponding to time and exploits ACC_{waist_y axis_t} as the variable of the column corresponding to time.

Meanwhile, the function F in Equation (2) may be defined in various forms. In an embodiment, the function F may be defined to calculate at least one of a geometric average, a minimum value, and a maximum value of the row x and the column y.

In an example, the function F may be defined by the following Equation (4) so as to calculate the geometric average of the row x and the column y.

F(x,y)=\sqrt{x²+y²} (4)

Therefore, based on the geometric average defined by Equation (4), the pixel value of a_{n-1,n} 301 illustrated in FIG. 5 may be calculated by the following Equation (5), using the x axis acceleration value of the behavior measurement device worn on the waist before an event occurrence time point (i.e., t=n−1) and the y axis acceleration value of the behavior measurement device worn on the waist at the event occurrence time point (t=1).

$${a}_{n - 1 , n} = F ( {x}_{n - 1} , {y}_{n} ) = \sqrt{{x}_{n - 1}^{2} + {y}_{n}^{2}} = \sqrt{{u ( t - 1 )}^{2} + {v ( t )}^{2}} = \sqrt{{( {Acc}_{waist , x axis , t = n - 1} )}^{2} + {( A c {c}_{waist , y axis , t = n} )}^{2}} \qquad ( 5 )$$

Meanwhile, referring to FIG. 6, at step S222 of converting the generated image tables into primary images according to an embodiment, image tables 310 to 330 for respective colors may be converted into primary images 311 to 313 in colors respectively corresponding thereto based on pixel values recorded in the image tables 310 to 330.

In accordance with an embodiment, step S223 of, when there are multiple primary images, generating one secondary image by combining respective primary images generated for two or more colors may be configured such that, if some of the behavior measurement devices 10-1, 10-2, . . . , 10-N are disconnected due to a power or communication problem or if some of the behavior measurement devices are not initially worn on the body, pieces of sensing data measured from one or two body regions may be converted into images.

For example, referring to FIG. 6, when only one of the behavior measurement devices 10-1, 10-2, . . . , 10-N transmits sensing data, a primary image generated based on the image table for the color corresponding to the one behavior measurement device may be determined to be a final image.

Further, as illustrated in FIG. 6, when only the behavior measurement device worn on the waist and the behavior measurement device worn on the right foot transmit sensing data, the final image may be generated by combining the red image table with the green image table. That is, as illustrated in FIG. 6, the patterns, shapes, and colors of generated images may completely differ from each other depending on the states of the behavior measurement devices 10-1, 10-2, . . . , 10-N. However, it is possible to analyze behavior according to an embodiment, just as it is possible to schematically identify whether the corresponding object is a dog or a person even if the corresponding image is represented by at least one of red, green, and blue.

Meanwhile, the value of each pixel in each image table according to an embodiment is characterized in that it is calculated using sensing data values at different time points, such as the time point n−1 before occurrence of the event and the event occurrence time point n, as shown in Equation (5), rather than being calculated using a sensing value at a single time point. That is, when the pixel values to be recorded in the image tables are calculated, the relationships between pieces of sensing data at different times can be calculated and reflected in pixel values. Accordingly, accurate behavior estimation results may be derived at the time of estimating the behavior of the user based on the learning model at the above-described step S230.

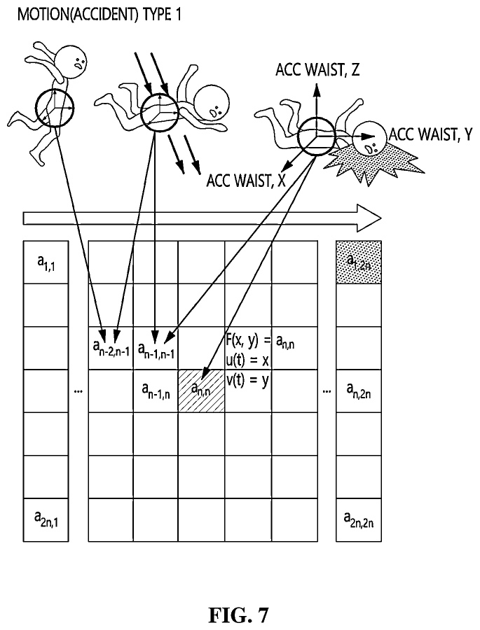

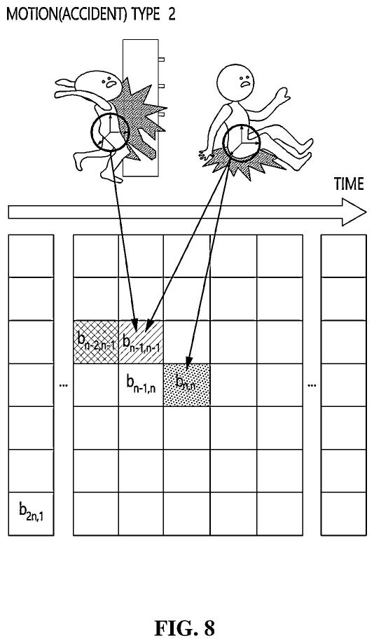

FIGS. 7 and 8 are diagrams illustrating examples of image tables when motion types are different from each other according to embodiments.

Referring to FIG. 7, in the case of motion (accident) type 1, at the time point n−1 before an event occurs, an acceleration of 1 g or less is measured in a waist region in free fall. Further, at the event occurrence time point n, an acceleration of 1 g or more is measured due to the impact caused by the event.

Meanwhile, referring to FIG. 8, in the case of motion (accident) type 2, at the time point n−1 before an event occurs, an acceleration greater than that at an event occurrence time point n is measured in a waist region while a pedestrian collides with the wall, and thereafter an event occurring while the pedestrian lands on the ground also has an acceleration greater than 1 g. Thus the image pattern of motion type 2 may be distinguished from that of motion type 1.

Further, unlike downward acceleration occurring in a forward direction at time points n−2 and n−1 in the case of motion type 1, illustrated in FIG. 7, upright-walking acceleration occurs in a forward direction in the case of motion type 2, illustrated in FIG. 8, and thus values calculated for a pixel at the same location may be different.

That is, referring to FIGS. 7 and 8, the patterns of images include information in a frequency domain, indicating how rapidly and greatly data values vary, and information obtained by digitizing relationships between respective axes and between regions, and thus the image patterns can be accurately and visually reflected in images.

Therefore, it is possible to precisely and accurately analyze the behavior of a pedestrian in all situations based on deep learning technology or image analysis technology by exploiting the images containing such information as input.

Meanwhile, as described above, at step S220 of converting the sensing data into the images, there may be an embodiment in which one behavior measurement device through which sensing data is acquired is present.

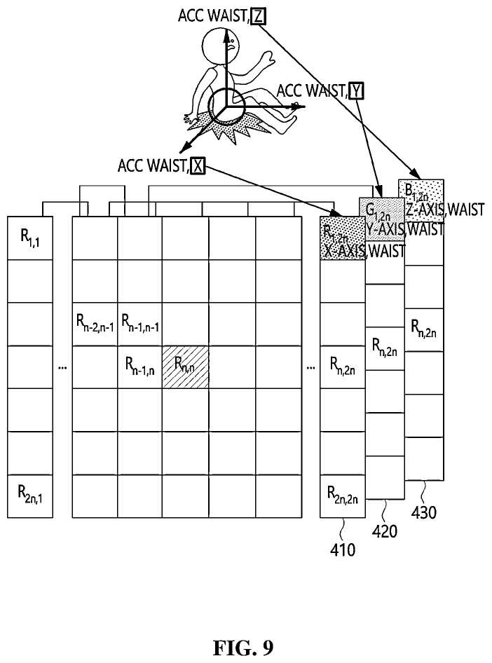

FIG. 9 is a diagram illustrating an example of 2D image tables according to another embodiment.

Referring to FIG. 9, when only a single behavior measurement device is worn on the waist of a user, the x axis acceleration value of the waist may be used to generate a red image table 410, the y axis acceleration value of the waist may be used to generate a green image table 420, and the z axis acceleration value of the waist may be used to generate a blue image table 430.

Therefore, respective pixel values in image tables, each composed of 2n×2n pixels from R_{1,1} to R_{2n,2n}, may be calculated in such a way that, for example, the pixel value of R_{n-1,n} is calculated based on a function that has the x axis acceleration value of the waist at a time point n−1 as a row x and the x axis acceleration value of the waist at a time point n as a column y and that has the row x and the column y as variables, such as those in Equation (5).

Further, as described above, at step S220 of converting the sensing data into images, there may be an embodiment in which an image converted from the sensing data is a 3D image.

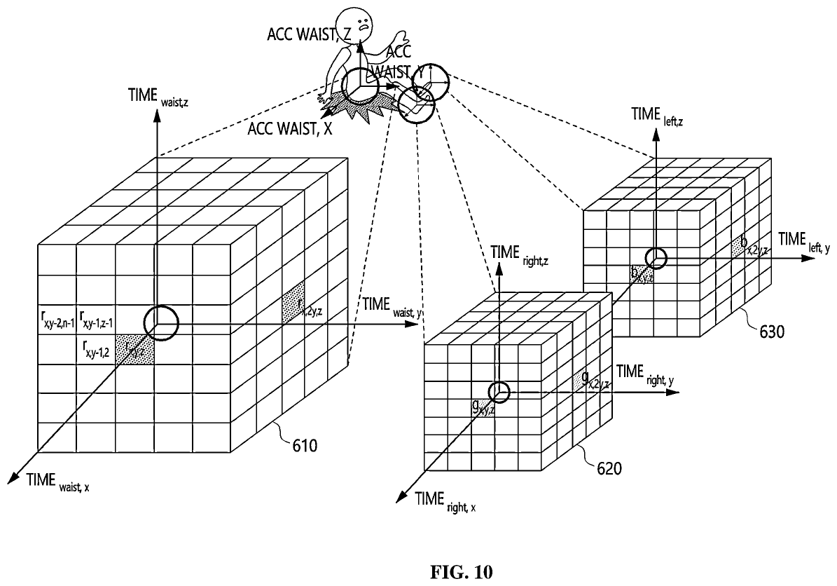

FIG. 10 is a diagram illustrating an example of 3D image tables according to a further embodiment.

Referring to FIG. 10, generation of a 3D image using the 3D image tables is similar to generation of a 2D image.

That is, as the 3D image tables, image tables corresponding to red, green and blue may be separately generated, similar to a 2D image generation method.

For example, referring to FIG. 10, when multiple behavior measurement devices 10-1, 10-2, . . . , 10-N are attached to the waist, left foot, and right foot, respectively, sensing data acquired through the behavior measurement device attached to the waist may be used to generate a red image table 610, sensing data acquired through the behavior measurement device attached to the right foot may be used to generate a green image table 620, and sensing data acquired through the behavior measurement device attached to the left foot may be used to generate a blue image table 630.

Further, each of the image tables may have a size of 2n×2n×2n with respect to an even occurrence time point n.

Meanwhile, three axes (row, column, and height) of each 3D image table denote time. Therefore, as represented by the following Equation (6), the pixel values may be calculated by substituting acceleration values on respective axes over time into a function F′. That is, similar to the 2D image table generation method, F′ may be defined in various forms. In an example, the following Equation (6) may be calculated so as to obtain the function F′ using geometric averages.

$${F}^{′} ( Ac {c}_{x} , {Acc}_{y} , {Acc}_{z} ) = \sqrt{{( A c {c}_{w a i s t , x axis} )}^{2} + {( A c {c}_{w a i st , y axis} )}^{2} + {( A c {c}_{w a i s t , z axis} )}^{2}} \qquad ( 6 )$$

$${F}^{′} ( A c {c}_{w a i s t , x} , Ac {c}_{w a i s t , y} , Ac {c}_{w a i s t , z} ) = {r}_{n , n , n}$$

$${F}^{′} ( {Acc}_{left foot , x} , {Acc}_{left foot , y} , {Acc}_{left foot , z} ) = {g}_{n , n , n}$$

$${F}^{′} ( {Acc}_{right foot , x} , {Acc}_{right foot , y} , {Acc}_{right foot , z} ) = {b}_{n , n , n}$$

In this way, three pieces of data on the same axis may be combined with each other, so that pieces of sensing data at different time points on the same axis may be combined with each other, and thus pixel values (r_{n,n,n}, g_{n,n,n}, b_{n,n,n}) may be calculated.

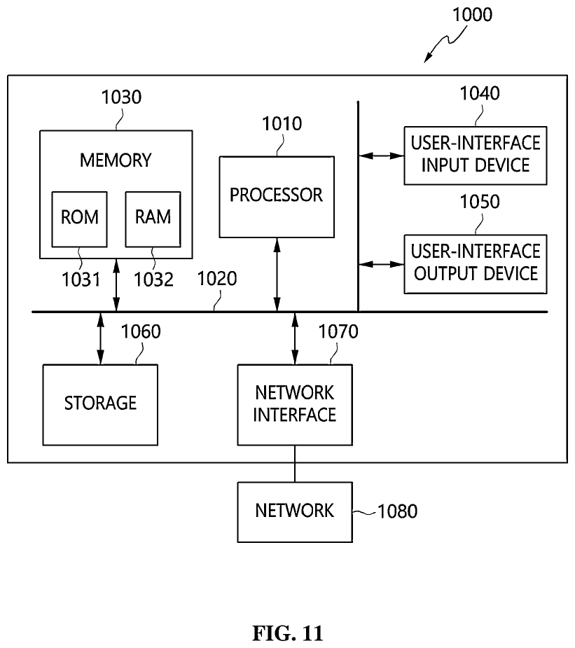

FIG. 11 is a diagram illustrating the configuration of a computer system according to an embodiment.

Each of an apparatus 20 for estimating the behavior of a user based on an image converted from sensing data (i.e., user behavior estimation apparatus 20) and a device (not illustrated) for converting sensing data into an image according to embodiments may be implemented in a computer system 1000 such as a computer-readable storage medium.

The computer system 1000 may include one or more processors 1010, memory 1030, a user interface input device 1040, a user interface output device 1050, and storage 1060, which communicate with each other through a bus 1020. The computer system 1000 may further include a network interface 1070 connected to a network 1080. Each processor 1010 may be a Central Processing Unit (CPU) or a semiconductor device for executing programs or processing instructions stored in the memory 1030 or the storage 1060. Each of the memory 1030 and the storage 1060 may be a storage medium including at least one of a volatile medium, a nonvolatile medium, a removable medium, a non-removable medium, a communication medium, or an information delivery medium. For example, the memory 1030 may include Read-Only Memory (ROM) 1031 or Random Access Memory (RAM) 1032.

In accordance with the embodiments, the behavior of a user may be accurately identified even in various environments and situations.

In accordance with the embodiments, the behavior of a user may be accurately identified depending on various patterns appearing in the same type of behavior.

Although the embodiments of the present invention have been disclosed with reference to the attached drawing, those skilled in the art will appreciate that the present invention can be implemented in other concrete forms, without changing the technical spirit or essential features of the invention. Therefore, it should be understood that the foregoing embodiments are merely exemplary, rather than restrictive, in all aspects.

What is claimed is:

1. An apparatus for estimating a behavior of a user based on an image converted from sensing data, comprising:
a memory for storing at least one program; and
a processor for executing the program,
wherein the program performs:
acquiring sensing data measured by one or more behavior measurement devices worn by the user;
converting sensing data of the user obtained for a predetermined time period into images; and
estimating the behavior of the user from the images of the user based on a pre-trained model.

2. The apparatus of claim 1, wherein the sensing data of the user obtained for the predetermined time period is measured during a predetermined time before and after a time point at which an event, an intensity of an impact of which is equal to or greater than a predetermined threshold value, occurred.

3. The apparatus of claim 1, wherein the program further performs:
upon converting the sensing data into the images,
generating a primary image for each of one or more colors based on the sensing data; and
when there are multiple primary images, generating one secondary image by combining primary images generated for each of two or more colors.

4. The apparatus of claim 3, wherein the program further performs:
upon generating the primary image for each of the one or more colors,
when there are multiple behavior measurement devices, generating image tables in which pixel values, calculated based on pieces of sensing data measured through respective multiple behavior measurement devices, are recorded; and
converting the generated image tables into primary images in different colors.

5. The apparatus of claim 3, wherein the program further performs:
upon generating the primary image for each of the one or more colors,
when there are multiple behavior measurement devices, generating multiple image tables in which pixel values, calculated by combining pieces of sensing data measured through the behavior measurement devices with each other, are recorded; and
converting the generated image tables into primary images in different colors.

6. The apparatus of claim 3, wherein:
the sensing data includes acceleration values on an x axis, a y axis, and a z axis over time for each of one or more behavior measurement devices worn on different body regions of the user, and
the program is configured to, upon generating the primary image for each of the one or more colors,
when each image is a two-dimensional (2D) image, convert a 2D image table into a primary image, wherein each pixel value of the 2D image table is determined to be any one of values calculated based on predetermined criteria that include a geometric average, a maximum value, and a minimum value of one or more of acceleration values on the x axis, the y axis, and the z axis over time, measured through the one or more behavior measurement devices.

7. The apparatus of claim 3, wherein:
the sensing data includes acceleration values on an x axis, a y axis, and a z axis over time for each of one or more behavior measurement devices worn on different body regions of the user, and
the program is configured to, upon generating the primary image for each of the one or more colors,
when each image is a three-dimensional (3D) image, convert a 3D image table into a primary image, wherein each pixel value of the 3D image table is determined to be a value calculated based on the acceleration values on the x axis, the y axis and the z axis over time, measured through the one or more behavior measurement devices.

8. The apparatus of claim 3, wherein the program further performs:
upon estimating the behavior of the user, determining based on the images whether the behavior of the user is in a normal or abnormal state; and
if it is determined that the behavior the user is in an abnormal state, reporting a dangerous situation.

9. A method for estimating a behavior of a user based on an image converted from sensing data, comprising:
acquiring sensing data measured by one or more behavior measurement devices worn by the user;
converting sensing data of the user obtained for a predetermined time period into images; and
estimating the behavior of the user from the images of the user based on a pre-trained model.

10. The method of claim 9, wherein the sensing data of the user obtained for the predetermined time period is measured during a predetermined time before and after a time point at which an event, an intensity of an impact of which is equal to or greater than a predetermined threshold value, occurred.

11. The method of claim 9, wherein converting the sensing data into the images comprises:
generating a primary image for each of one or more colors based on the sensing data; and
when there are multiple primary images, generating one secondary image by combining primary images generated for each of two or more colors.

12. The method of claim 11, wherein generating the primary image for each of the one or more colors comprises:
when there are multiple behavior measurement devices, generating image tables in which pixel values, calculated based on pieces of sensing data measured through respective multiple behavior measurement devices, are recorded; and
converting the generated image tables into primary images in different colors.

13. The method of claim 11, wherein generating the primary image for each of the one or more colors comprises:
when there are multiple behavior measurement devices, generating multiple image tables in which pixel values, calculated by combining pieces of sensing data measured through the behavior measurement devices with each other, are recorded; and
converting the generated image tables into primary images in different colors.

14. The method of claim 11, wherein:
the sensing data includes acceleration values on an x axis, a y axis, and a z axis over time for each of one or more behavior measurement devices worn on different body regions of the user, and
generating the primary image for each of the one or more colors is configured to, when each image is a two-dimensional (2D) image, convert a 2D image table into a primary image, wherein each pixel value of the 2D image table is determined to be any one of a geometric average, a maximum value, and a minimum value of one or more of acceleration values on the x axis, the y axis, and the z axis over time, measured through the one or more behavior measurement devices.

15. The method of claim 11, wherein:
the sensing data includes acceleration values on an x axis, a y axis, and a z axis over time for each of one or more behavior measurement devices worn on different body regions of the user, and
generating the primary image for each of the one or more colors is configured to, when each image is a three-dimensional (3D) image, convert a 3D image table into a primary image, wherein each pixel value of the 3D image table is determined to be a value calculated based on the acceleration values on the x axis, the y axis and the z axis over time, measured through the one or more behavior measurement devices.

16. A method for converting sensing data into an image, comprising:
generating a primary image for each of one or more colors based on sensing data of a user obtained for a predetermined time period; and
when there are multiple primary images, generating one secondary image by combining primary images generated for each of two or more colors.

17. The method of claim 16, wherein generating the primary image for each of one or more colors comprises:
when the sensing data is acquired from multiple behavior measurement devices, generating image tables in which pixel values, calculated based on pieces of sensing data measured through respective multiple behavior measurement devices, are recorded; and
converting the generated image tables into primary images in different colors.

18. The method of claim 16, wherein generating the primary image for each of one or more colors comprises:
when the sensing data is acquired from multiple behavior measurement devices, generating multiple image tables in which pixel values, calculated by combining pieces of sensing data measured through the behavior measurement devices with each other, are recorded; and
converting the generated image tables into primary images in different colors.

19. The method of claim 16, wherein:
the sensing data includes acceleration values on an x axis, a y axis, and a z axis over time for each of one or more behavior measurement devices worn on different body regions of the user, and
generating the primary image for each of the one or more colors is configured to, when each image is a two-dimensional (2D) image, convert a 2D image table into a primary image, wherein each pixel value of the 2D image table is determined to be any one of a geometric average, a maximum value, and a minimum value of one or more of acceleration values on the x axis, the y axis, and the z axis over time, measured through the one or more behavior measurement devices.

20. The method of claim 16, wherein:
the sensing data includes acceleration values on an x axis, a y axis, and a z axis over time for each of one or more behavior measurement devices worn on different body regions of the user, and
generating the primary image for each of the one or more colors is configured to, when each image is a three-dimensional (3D) image, convert a 3D image table into a primary image, wherein each pixel value of the 3D image table is determined to be a value calculated based on the acceleration values on the x axis, the y axis and the z axis over time, measured through the one or more behavior measurement devices.
